(19) United States
(12) Patent Application Publication
(10) Pub. No.: US 2010/0114149 A1
(43) Pub. Date: May 6, 2010

(54) AUTOMATICALLY ADJUSTING INTRA-GASTRIC SATIATION AND SATIETY CREATION DEVICE

(76) Inventors: Thomas E. ALBRECHT, Cincinnati, OH (US); Jason L. HARRIS, Mason, OH (US); Mark S. ORTIZ, Milford, OH (US); Michael J. STOKES, Cincinnati, OH (US); Mark S. ZEINER, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,084

(22) Filed: Oct. 30, 2008

(51) Int. Cl. A61M 29/02; A61B 5/107; F04B 17/03

(52) U.S. Cl. 606/192; 600/593; 417/410.1

(57) ABSTRACT

An implant for placement within a hollow body organ, the implant includes a distension device having an undeployed shape for delivery within a hollow body and one or more deployed shapes for implantation therein. The device has sufficient rigidity in its deployed shape to exert an outward force against an interior of the hollow body so as to bring together two substantially opposing surfaces of the hollow body. The implant further includes an implantable pump in fluid communication with the distension device and having a plurality of actuators configured to change the shape of the distension device upon the application of energy thereto such that sequential activation of the plurality of actuators is effective to create pumping action to move fluid through the pump.

### FIELD OF THE INVENTION

The present invention relates to implantable medical devices, and in particular implantable gastric distension devices.

### BACKGROUND OF THE INVENTION

Obesity is becoming a growing concern, particularly in the United States, as the number of obese people continues to increase, and more is learned about the negative health effects of obesity. Morbid obesity, in which a person is 100 pounds or more over ideal body weight, in particular poses significant risks for severe health problems. Accordingly, a great deal of attention is being focused on treating obese patients. One proposed method of treating morbid obesity has been to place a distension device, such as a, spring loaded coil inside the stomach. Examples of satiation and satiety inducing gastric implants, optimal design features, as well as methods for installing and removing them are described in commonly owned and pending U.S. patent application Ser. No. 11/469,564, filed Sep. 1, 2006, and pending U.S. patent application Ser. No. 11/469,562, filed Sep. 1, 2006, which are hereby incorporated herein by reference in their entirety. One effect of the coil is to more rapidly induce feelings of satiation defined herein as achieving a level of fullness during a meal that helps regulate the amount of food consumed. Another effect of the coil is to prolong the effect of satiety which is defined herein as delaying the onset of hunger after a meal which in turn regulates the frequency of eating. By way of a non-limiting list of examples, positive impacts on satiation and satiety may be achieved by an intragastric coil through one or more of the following mechanisms: reduction of stomach capacity, rapid engagement of stretch receptors, alterations in gastric motility, pressure induced alteration in gut hormone levels, and alterations to the flow of food either into or out of the stomach.

With each of the above-described food distension devices, safe, effective treatment requires that the device be regularly monitored and adjusted to vary the degree of distension applied to the stomach.

During these gastric coil adjustments, it may be difficult to determine how the adjustment is proceeding, and whether the adjustment will have the intended effect. In an attempt to determine the efficacy of an adjustment, some physicians may utilize fluoroscopy with a Barium swallow as the adjustment is being performed. However, fluoroscopy is both expensive and undesirable due to the radiation doses incurred by both the physician and patient. A, a physician may simply adopt a “try as you go” method based upon their prior experience, and the results of an adjustment may not be discovered until hours or days later, when the patient experiences too much distension to the stomach cavity, or the coil induces erosion of the stomach tissue due to excessive interface pressures against the coil.

Furthermore, the implantable pumps known in the art, such as centrifugal or positive displacement pumps, have high power requirements during operation. The power requirements of such pumps limit their usage for frequent adjustments to fluid levels in the coil. Current pumps also require large housings to encase the mechanical pumping mechanism, gears, and motors, further limiting their usefulness as implantable pumps. Additional components, such as valves, are also necessary to maintain fluid pressure in the coil when power is not supplied to conventional pumps. An example of an implantable pump system is described in US Patent Publication No. 2005/0277974, entitled “Thermodynamically driven reversible infuser pump for use as a remotely controlled gastric band” which was filed on May 28, 2004.

Accordingly, methods and devices are provided for use with an gastric distension device, and in particular methods and devices are provided which allow adjustment of an gastric distension device.

### BRIEF DESCRIPTION OF THE DRAWINGS

The invention will be more fully understood from the following detailed description taken in conjunction with the accompanying drawings, in which:

FIG. 1A is a schematic diagram of an embodiment of a stomach distension system;

FIG. 1B is a perspective view of an embodiment of an implantable portion of the stomach distension system of FIG. 1A;

FIG. 2A is a perspective view of the stomach distension device of FIG. 1A;

FIG. 2B is a schematic diagram of the stomach distension device of FIG. 2A applied about the gastro-esophageal junction of a patient;

FIG. 3 is a perspective view of an embodiment of the injection port housing of FIG. 1A;

FIG. 4 is a perspective view of an embodiment of the sensor housing of FIG. 1A;

FIG. 5 is a perspective view of an implantable portion of the stomach distension system according to one embodiment of the invention.

FIG. 6A is a perspective view of one exemplary embodiment of a pump having multiple actuators disposed around a flexible tube;

FIG. 6B is a perspective view of the pump of FIG. 6A with the first actuator activated;

FIG. 6C is a perspective view of the pump of FIG. 6A with the first and second actuators activated;

FIG. 6D is a perspective view of the pump of FIG. 6A with the first actuator deactivated and the second actuator activated;

FIG. 6E is a perspective view of the pump of FIG. 6A with the second and third actuators activated;

FIG. 6F is a perspective view of the pump of FIG. 6A with the second actuator deactivated and the third actuator activated;

FIG. 6G is a perspective view of the pump of FIG. 6A with the third and fourth actuators activated;

FIG. 7 is a perspective view of one exemplary embodiment of a pump having multiple actuators disposed around a flexible tube including a reaction surface;

FIG. 8 is a perspective view of one exemplary embodiment of a pump having multiple actuators disposed within a tubular member;

FIG. 9 is a perspective view of one exemplary embodiment of a pump having actuators disposed both around and within a flexible tubular member.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides systems and methods for forming a distension in a patient. In one exemplary embodiment, a distension system includes an implantable distension device and an implantable pump in fluid communication with the distension device. Optionally, an implantable port can be in fluid communication with the implantable distension device and the pump. The implantable distension device is adjustable and configured to form a distension in a patient, and the implantable port, if present, is configured to receive fluid from a fluid source external to the patient. Exemplary non-limiting examples of adjustable implantable distension devices (e.g., satiation and satiety inducing gastric implants), optimal design features, as well as methods for installing and removing them are described in commonly owned and pending U.S. patent application Ser. No. [ ], filed on even date herewith and entitled “Devices and Methods for Adjusting a Satiation and Satiety-Inducing Implanted Device” [Atty. Docket No. END6514USNP], which is hereby incorporated herein by reference in its entirety. The implantable pump has a plurality of actuators configured to change shape upon the application of energy thereto such that sequential activation of the plurality of actuators is effective to create pumping action to move fluid through the pump. Fluid in the distension system can move in a direction from the pump to the distension device or in a direction from the distension device to the pump. In one embodiment, the pump can be in fluid communication with the implantable port. The system can also include an implantable sensor in communication with the distension device and configured to measure at least a pressure within the distension device. The distension system can optionally include a fluid reservoir in fluid communication with the pump. The fluid reservoir is configured to hold fluid and can be configured to hold in the range of approximately 0.1 to 20 ml of fluid.

The implantable pump and its plurality of actuators can be arranged in a variety of configurations. In one exemplary embodiment the pump includes a first member having a passageway formed therethrough and being in communication with the plurality of actuators. The actuators can be disposed within the first member or outside the first member. At least one actuator can be configured to expand or contract (e.g., radially or axially) upon the application of energy thereto, and each of the actuators can be configured to move sequentially or independently. In one embodiment, at least one of the actuators can serve as a valve that is able to selectively control the passage of fluid by permitting, preventing, or limiting the passage of fluid. In one exemplary embodiment each actuator comprises an electroactive polymer.

The pump can be manually activated to move fluid either toward or away from the distension device. Alternatively, the pump can be automatically activated, such as by techniques including timer control, or programmed to be activated in response to certain sensed parameters.

In one embodiment, the implantable pump effects a pressure change within the distension device in accordance with a programmed schedule.

Further disclosed herein are methods for adjusting pressure in an implantable distension device. In one embodiment, the method can include sensing a clinically relevant parameter, adjusting a pressure within the distension device in response to the sensed clinically relevant parameter by activating a pump in fluid communication with the distension device. In one embodiment, the pump can be formed of a plurality of actuators configured to change shape upon the application of energy thereto such that sequential activation of the plurality of actuators is effective to create pumping action to move fluid through the pump. The sensing of the clinically relevant parameter can be effected using an implantable sensor. The clinically relevant parameter can be a pressure, in which case, the implantable sensor is a pressure sensor. In such an embodiment, the sensed pressure is compared to a desired pressure range and the pressure within the distension device is adjusted to be approximately within the desired pressure range if the sensed pressure is not within a desired pressure range. In one embodiment, the pump can be automatically activated, although other activation techniques, including manual activation, are also envisioned.

Also disclosed herein is a pumping device including a fluid conduit member having a passageway formed therethrough, and a plurality of orientation-changing actuators disposed within the fluid conduit member. Each actuator is independently configurable between a normal, relaxed state in which the actuator occludes a portion of the passageway of the fluid conduit member and an energized configuration in which fluid flow is permitted between an outer surface of the actuator and an inner surface of the fluid conduit member. The actuators are also configured to change orientation upon the application of energy thereto such that sequential activation of the plurality of actuators is effective to create pumping action to move fluid through the first member.

The actuators can be formed from a variety of materials. In one exemplary embodiment, each actuator comprises an electroactive polymer (EAP). For example, each actuator can include a least one electroactive polymer composite having at least one flexible conductive layer, an electroactive polymer layer, and an ionic gel layer. Each actuator can also include a return electrode and a delivery electrode coupled thereto, the delivery electrode being adapted to deliver energy from an energy source. The actuators can be configured to move independently or sequentially.

The present invention generally provides systems and methods for forming a distension in a patient. In general, the systems and methods allow the pressure or volume of fluid in a distension device to be adjusted. The pressure or volume adjustment is effected by the use of an implantable pump. The implantable pump allows the pressure or volume of fluid in a distension device to be adjusted without the need for fluid to be added from an external source.

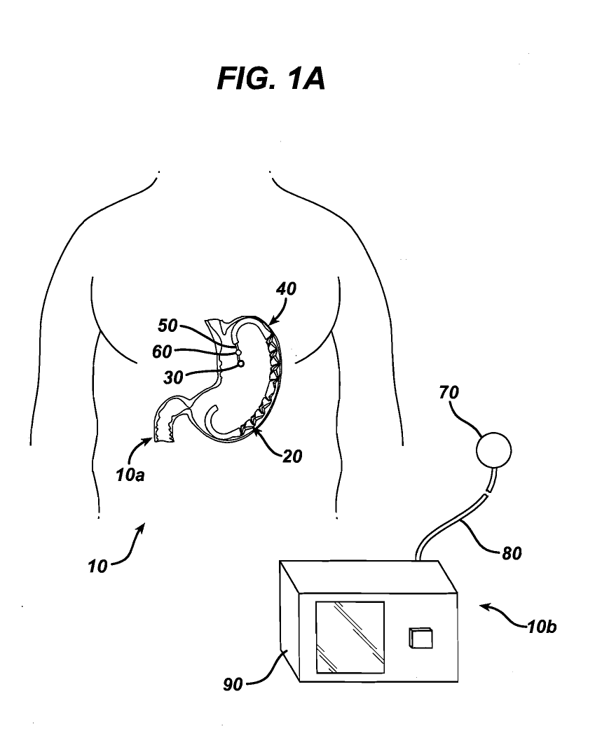

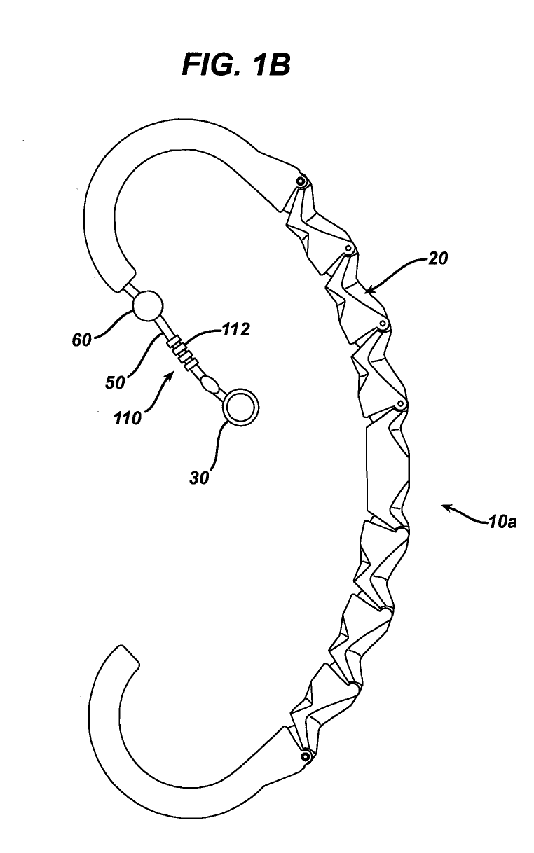

While the present invention can be used with a variety of distension systems known in the art, FIG. 1A illustrates one exemplary embodiment of a stomach distension system 10 in use in a patient. As shown, the system 10 generally includes an implantable portion 10a and an external portion 10b. FIG. 1B illustrates the implantable portion 10a outside of a patient. The implantable portion 10a includes an adjustable gastric coil 20 that is configured to be positioned around the upper portion of a patient's stomach 40, and an injection port housing 30 that is fluidly coupled to the adjustable gastric coil 20, e.g., via a catheter 50.

The injection port housing 30 is adapted to allow fluid to be introduced into and removed from the gastric coil 20 to thereby adjust the size of the coil, and thus the pressure applied to the stomach. The injection port housing 30 can thus be implanted at a location within the body that is accessible through the tissue. Typically, injection ports may be positioned on the coil or attached to a layer of the stomach

The internal portion 10a can also include a sensing or measuring device in fluid communication with the closed fluid circuit in the implantable portion 10a such that the measuring device can take measurements related to any parameter relevant to implantable distension devices. Such clinically relevant parameters include, but are not limited to, temperature, pressure, changes in pressure, acoustic input, tissue impedance, changes in sensed tissue impedance, chemical composition, changes in chemical composition, pulse count, pulse width and amplitude. While the methods and devices discussed herein can relate to any sensed data parameter, in an exemplary embodiment, the measurements relate to pressure, and the methods and devices disclosed herein will be discussed in the context of measuring the fluid pressure of the closed fluid circuit. While the measuring device can have various configurations and it can be positioned anywhere along the internal portion 10a, including within the injection port housing 30, in the illustrated embodiment the measuring device is in the form of a pressure sensor that is disposed within a sensor housing 60 positioned adjacent to the injection port housing 30. The catheter 50 can include a first portion that is coupled between the gastric coil 20 and the sensor housing 60, and a second portion that is coupled between the sensor housing 60 and the injection port housing 30.

In addition to sensing pressure of fluid within the internal portion 10a, pressure of fluid within the esophagus and/or the stomach 40 can also be sensed using any suitable device, such as an endoscopic manometer. By way of non-limiting example, such fluid pressure measurements can be compared against measured pressure of fluid within the internal portion 10a before, during, and/or after adjustment of pressure within the internal portion 10a. Other suitable uses for measured pressure within the esophagus and/or the stomach 40 will be appreciated by those skilled in the art.

As further shown in FIG. 1A, the external portion 10b generally includes a pressure reading device 70 that is configured to be positioned on the skin surface above the sensor housing 60 (which can be implanted beneath thick tissue, e.g., over 10 cm thick) to non-invasively communicate with the sensor housing 60 and thereby obtain pressure measurements. The pressure reading device 70 can optionally be electrically coupled (in this embodiment via an electrical cable assembly 80) to a control box 90 that can display the pressure measurements, or other data obtained from the pressure reading device 70.

FIG. 1B shows the implantable portion 10a in more detail. In the illustrated embodiment, the implantable portion 10a includes an adjustable gastric coil 20, an injection port housing 30 that is fluidly coupled to the adjustable gastric coil 20, a sensor housing 60, and a pump 110. The pump 110 can have a variety of configurations which will be discussed in more detail below. In the embodiment shown in FIG. 1B, the pump 110 generally includes an elongate member 112.

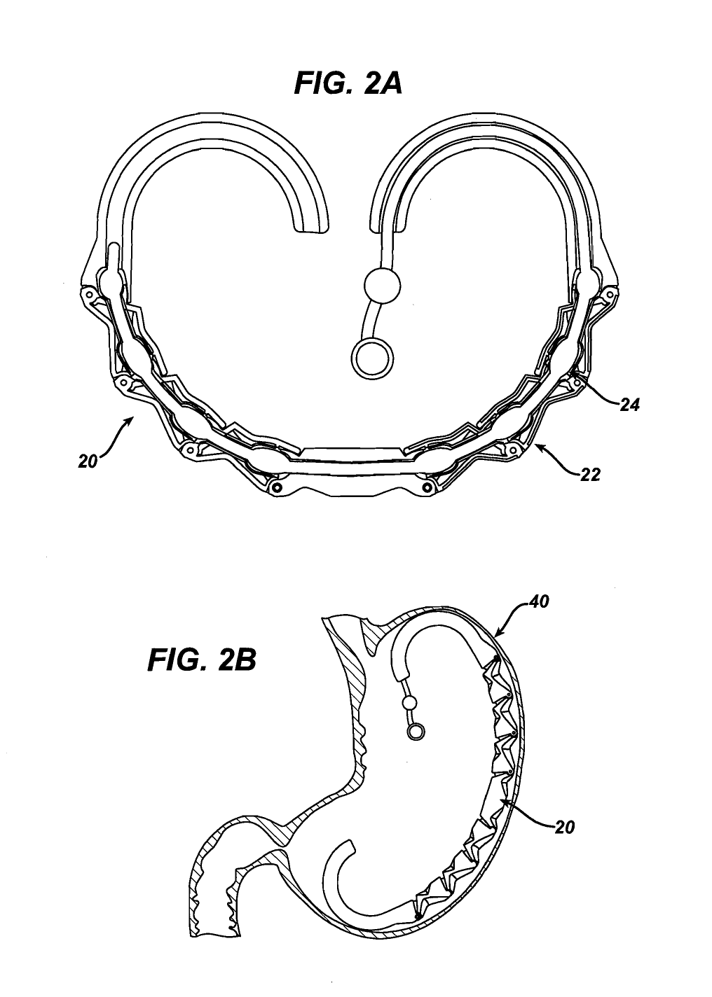

FIG. 2A shows the gastric coil 20 in more detail. While the gastric coil 20 can have a variety of configurations, and various gastric coils currently known in the art can be used with the present disclosure, in the illustrated embodiment the gastric coil 20 has a generally elongate shape with a support structure 22 having first and second opposite ends 20a, 20b that can be formed in a C-shape. Various techniques can be used to keep the ends 20a, 20b in relative proximity to one another. In the illustrated embodiment, the fluid bladder pressure may be varied to control the proximity of the ends relative to each other. The gastric coil 20 can also include a variable volume member, such as an inflatable balloon 24, that is disposed or formed on one side of the support structure 22 and that is configured to be positioned adjacent to tissue. The balloon 24 can expand or contract against the outer wall of the coil to form an adjustable size coil for controllably restricting food intake into the stomach.

A person skilled in the art will appreciate that the gastric coil can have a variety of other configurations. Moreover, the various methods and devices disclosed herein have equal applicability to other types of implantable coils.

FIG. 2B shows the adjustable gastric coil 20 applied the stomach of a patient. As shown, the coil 20 at least substantially distends the stomach 40. After the coil 20 is implanted, it may be deployed. A person skilled in the art will appreciate that various techniques, including mechanical and electrical techniques, can be used to adjust the coil.

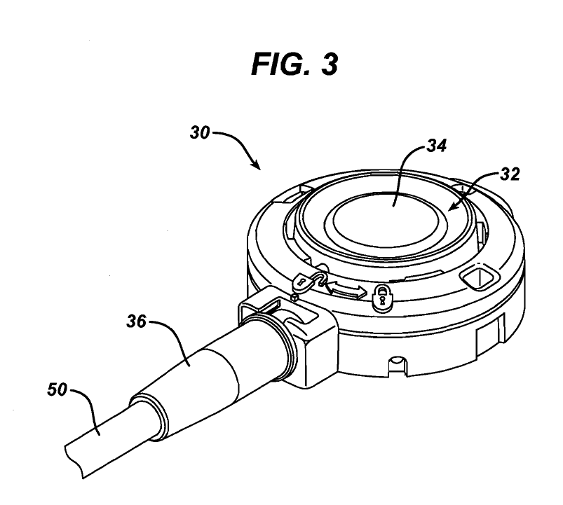

The fluid injection port housing 30 can also have a variety of configurations. In the embodiment shown in FIG. 3, the injection port housing 30 has a generally cylindrical shape with a distal or bottom surface and a perimeter wall extending proximally from the bottom surface and defining a proximal opening 32. The proximal opening 32 can include a needle-penetrable septum 34 extending there across and providing access to a fluid reservoir (not visible in FIG. 3) formed within the housing. The septum 34 is preferably placed in a proximal enough position such that the depth of the reservoir is sufficient enough to expose the open tip of a needle, such as an endoscopic Huber-like needle, so that fluid transfer can take place. The septum 34 is preferably arranged so that it will self seal after being punctured by a needle and the needle is withdrawn. As further shown in FIG. 3, the housing 30 can further include a catheter tube connection member 36 that is in fluid communication with the reservoir and that is configured to couple to a catheter (e.g., the catheter 50). A person skilled in the art will appreciate that the housing can be made from any number of materials, including stainless steel, titanium, or polymeric materials, and the septum 34 can likewise be made from any number of materials, including silicone.

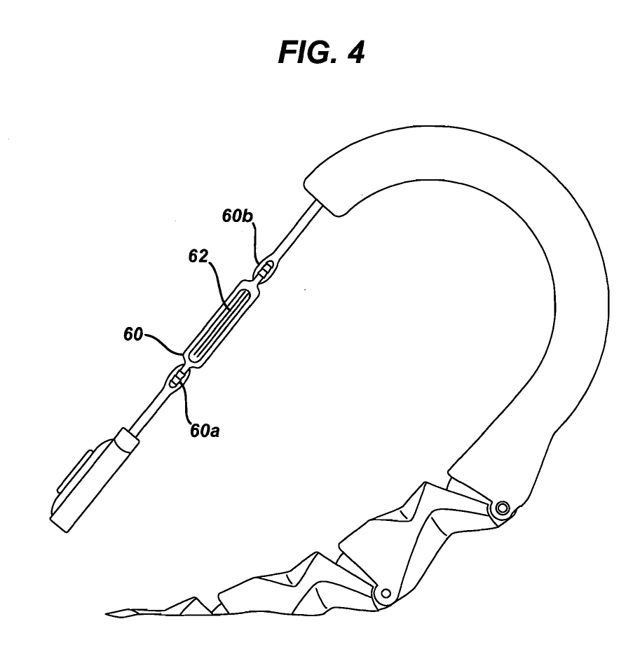

As indicated above, the system 10 can also include a pressure measuring device in communication with the closed fluid circuit and configured to measure pressure (e.g., fluid pressure) which corresponds to the amount of distension applied by the adjustable gastric coil 20 to the patient's stomach 40. Measuring the pressure enables a person (e.g., a physician, a nurse, a patient, etc.) to evaluate the efficacy and functionality of the distension created by a coil adjustment. In the illustrated embodiment, as shown in FIG. 4, the pressure measuring device is in the form of a pressure sensor 62 disposed within the sensor housing 60. The pressure measuring device can, however, be disposed anywhere within the closed hydraulic circuit of the implantable portion.

In general, the illustrated sensor housing 60 includes an inlet 60a and an outlet 60b that are in fluid communication with the fluid in the implantable portion 10a. An already-implanted catheter 50 can be retrofitted with the sensor housing 60, such as by severing the catheter 50 and inserting barbed connectors (or any other connectors, such as clamps, clips, adhesives, welding, etc.) into the severed ends of the catheter 50. The sensor 62 can be disposed within the housing 60 and be configured to respond to fluid pressure changes within the hydraulic circuit and convert the pressure changes into a usable form of data. The pressure sensor 62 disposed within the housing 60 can sense and monitor the adjusted state of the coil statically or while fluid is being pumped.

While not shown, the pressure sensing system can also include a microcontroller, a TET/telemetry coil, and a capacitor. Optionally, the pressure sensing system can further comprise a temperature sensor (not shown). The microcontroller, TET/telemetry coil, and capacitor can be in communication via a circuit board (not shown) or any via any other suitable component(s). It will also be appreciated that TET/telemetry coil and capacitor may collectively form a tuned tank circuit for receiving power from external portion, and transmitting the pressure measurement to the pressure reading device.

Various pressure sensors known in the art can be used, such as a wireless pressure sensor provided by CardioMEMS, Inc. of Atlanta, Ga., though a suitable MEMS pressure sensor may be obtained from any other source, including but not limited to Integrated Sensing Systems (ISSYS), and Remon Medical. One exemplary MEMS pressure sensor is described in U.S. Pat. No. 6,855,115, the disclosure of which is incorporated by reference herein for illustrative purposes only. It will also be appreciated that suitable pressure sensors may include, but are not limited to, capacitive, piezoresistive, silicon strain gauge, or ultrasonic (acoustic) pressure sensors, as well as various other devices capable of measuring pressure. Additionally an angle measurement may be used where the angle between any of the individual links is measured with a one of the above mentioned methods.

The pressure reading device 70 can also have a variety of configurations, and one exemplary pressure reading device is disclosed in more detail in commonly-owned U.S. Patent Application Publication No. 2006/0189888 and U.S. Patent Application Publication No. 2006/0199997, each of which is hereby incorporated by reference in its entirety. In general, the pressure reading device 70 can non-invasively measure the pressure of the fluid within implanted portion even when the injection port housing 30 or sensor housing 60 is implanted beneath thick (at least over 10 centimeters) subcutaneous fat tissue. The physician may hold pressure-reading device 70 against the patient's skin near the location of sensor and observe the pressure reading on a display on the control box 90. The pressure reading device 70 can also be removably attached to the patient, such as during a prolonged examination, using straps, adhesives, and other well-known methods. The pressure reading device 70 can operate through conventional cloth or paper surgical drapes, and can also include a disposable cover (not shown) that may be replaced for each patient.

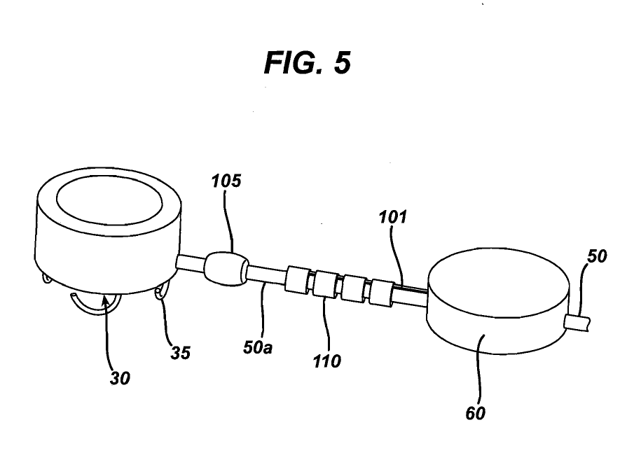

FIG. 5 illustrates one embodiment of the proximal end of the implantable portion 10a (FIGS. 1A and 1B) of the implantable distension system 10. As shown, the proximal end of the implantable portion 10a includes an injection port housing 30, which is in fluid communication with a reservoir 105 and a pump 110. The proximal end may also include a sensor housing 60, as well as one or more sensor/power leads 101. Conduit 50a provides fluid communication between the individual components of the proximal end of the implantable portion 10a. Catheter 50 provides fluid communication between the proximal end of the implantable portion 10a shown in FIG. 5 and downstream distension device 20 (FIG. 1B). Although the components shown in FIG. 5 are shown in an inline configuration, one skilled in the art will appreciate that the components can be connected in any order and in any configuration, i.e., in a T configuration or a Y configuration, for example.

As shown in FIG. 5, the injection port housing 30, if present, can optionally include an anchoring device, such as hooks 35, that can be used to anchor the injection port housing 30 within the patient's body, preferably to the inner surface of the stomach. Although FIG. 5 shows that the housing 30 is arranged in line with the reservoir 105, the pump 110 and the sensor housing 60, the housing 30 can be connected to the other components and conduit 50a in other ways, i.e., in a T configuration or a Y configuration, for example. The injection port housing 30 itself is optional because the implantable distension system 10a (FIG. 1B) can be filled with fluid prior to implantation or at the time of implantation. The pressure in the downstream distension device 20 (FIG. 1B) can then be adjusted using the pump 110 to move fluid into or out of the distension device 20.

Reservoir 105 provides an optional means for holding an additional supply of fluid. For example, the reservoir 105 can contain 0.1-20 ml of fluid. As shown, the reservoir 105 can be a portion of conduit 50a with a larger diameter than the nominal diameter of the conduit 50a. Various other configurations can be used to provide a reservoir 105, such as separate reservoir components connected to, and in fluid communication with, the conduit 50a or any other components, i.e., the injection port housing 30, the pump 110 or the sensor housing 60. Although FIG. 5 shows that the reservoir 105 is arranged in line between the pump 110 and the injection port housing 60, one skilled in the art will appreciate that the reservoir 105 can be connected to the other components and conduit 50a in other ways, i.e., in a T configuration or a Y configuration, for example. It will also be appreciated that the reservoir 105 need not necessarily contain enough fluid to fill and empty the entire coil 20 (FIGS. 1A and 1B). For example, during the first fills of the coil 20, fluid may be delivered via an injection through the injection port housing 30. During this time the pump 110 can be retained in an open position. Alternatively, the reservoir 105 can be filled and then the fluid can be delivered to the coil 20 by the pump 110. Once the coil 20 is at functional fullness, i.e., occluding the stomach enough to cause a distension of intake, the reservoir 105 can be filled with enough fluid to accommodate future fill and adjustment needs without the need to add additional fluid via an injection port housing 30. One skilled in the art will appreciate that the reservoir 105 is optional, and in an embodiment without reservoir 105, not shown, the conduit 50a can optionally contain enough fluid to allow adjustments to the amount of fluid in the coil 20.

The embodiment shown in FIG. 5 includes an optional sensor housing 60 that is disposed in fluid communication with the components of the proximal end of the implantable portion 10a (FIG. 1B). Although FIG. 5 shows that the sensor housing 60 is arranged inline with the catheter 50 and the conduit 50a, one skilled in the art will appreciate that the sensor housing 60 can be connected to the other components in other ways, i.e., in a T configuration or a Y configuration, for example. Alternatively, a sensor can be placed in other locations in the system, such as on the coil itself. Sensor/power leads 101 can provide a connection between the sensor housing 60 and the pump 110 to supply energy to the pump, as will be discussed in more detail below.

The implantable pump 110 functions to move fluid into and out of the coil 20 to increase or decrease pressure within the coil as needed. Although the pump can have a variety of configurations, in one example the pump is based upon electroactive polymer (EAP) technology as discussed in more detail below. The use of EAP technology to form an implantable pump 110 provides a number of advantages, such as small size, low voltage requirements, high power density, and simplicity in terms of the number of moving parts.

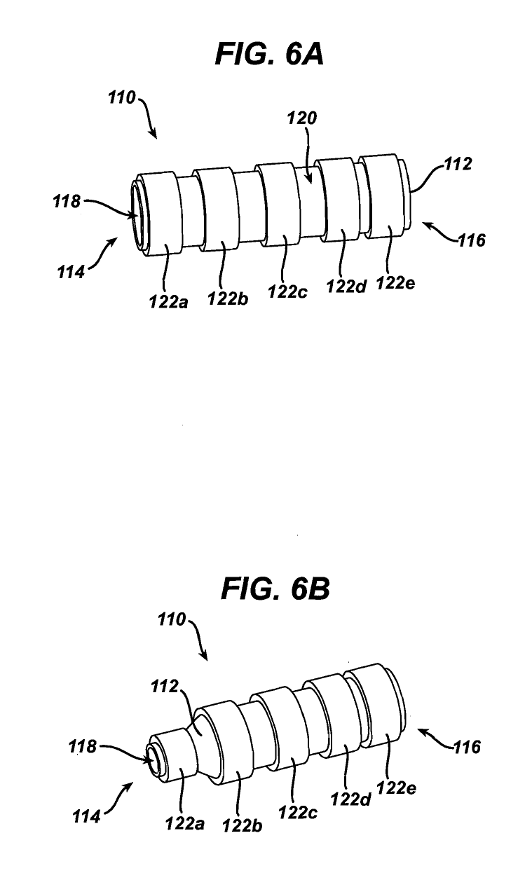

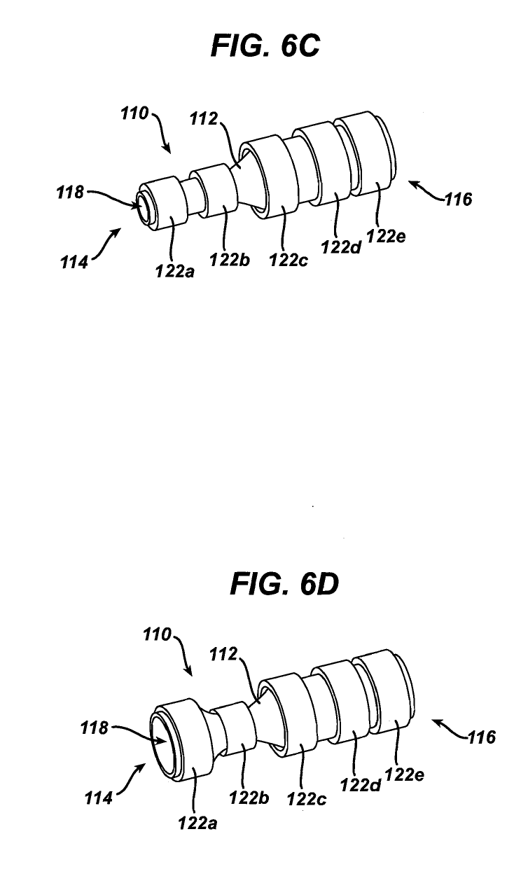

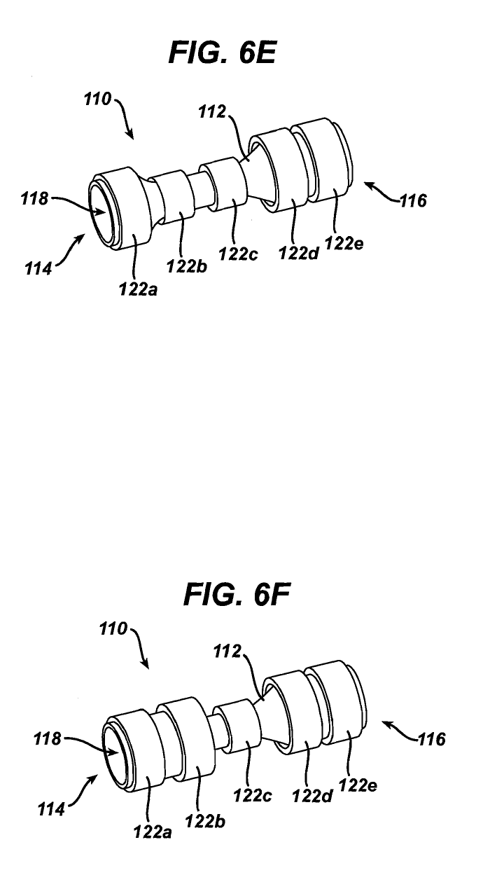

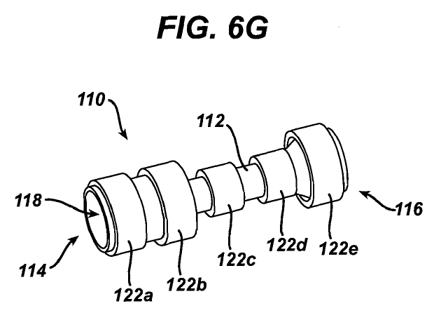

FIG. 6A illustrates one exemplary embodiment of a pumping mechanism using EAP actuators. As shown, the pump 110 generally includes an elongate member 112 having a proximal end 114, a distal end 116, and an inner passageway or lumen 118 extending therethrough between the proximal and distal ends 114, 116. The inner lumen 118 defines a fluid pathway. The elongate member 112 may optionally be formed as a portion of conduit 50a (FIG. 5) such that the pump 110 is in fluid communication with catheter 50 and downstream distension device 20. As shown, the pump 110 can include multiple actuators 122a, 122b, 122c, 122d, 122e that are disposed around the outer surface 120 of the elongate member 112. In use, as will be explained in more detail below, the actuators 122a-122e can be sequentially activated using electrical energy to cause the actuators 122a-122e to radially contract, thereby contracting the elongate member 112 and forcing fluid to move in one direction therethrough. The actuators can also be configured to axially contract and expand to move fluid through the elongate member 112.

In an alternative embodiment, the expansion and contraction of actuators 122a-122e may be used only to increase or decrease internal pressure within the coil without actually moving fluid. In this embodiment, the pump 110 could be disposed anywhere in fluid communication with the distension system. Upon the application of energy to the one or more of the actuators 122a-122e, the volume of the inner lumen 118 would be changed, effecting a corresponding pressure change in the system 10.

The elongate member 112 can have a variety of configurations, but in one exemplary embodiment it is in the form of a flexible elongate tube or cannula that is configured to receive fluid flow therethrough, and that is configured to flex and/or change size in response to orientational changes in the actuators 122a-22e. The shape and size of the elongate member 112, as well as the materials used to form a flexible and/or elastic elongate member 112, can vary depending upon the intended use. In certain exemplary embodiments, the elongate member 112 can be formed from a biocompatible polymer, such as silicone or latex. Other suitable biocompatible elastomers include, by way of non-limiting example, synthetic polyisoprene, chloroprene, fluoroelastomer, nitrile, and fluorosilicone. A person skilled in the art will appreciate that the materials can be selected to obtain the desired mechanical properties. While not shown, the elongate member 112 can also include other features to facilitate attachment thereof to a medical device, a fluid source, etc.

The actuators 122a-122e can also have a variety of configurations. In the illustrated embodiment, the actuators 122a-122e are formed into an annular member from an EAP laminate or composite that is rolled around an outer surface 120 of the elongate member 112. An adhesive or other mating technique can be used to attach the actuators 122a-122e to the elongate member 112. The actuators 122a-122e are preferably spaced a distance apart from one another to allow the actuators 122a-122e to radially contract and axially expand when energy is delivered thereto, however they can be positioned in contact with one another. A person skilled in the art will appreciate that actuators 122a-122e can alternatively be disposed within the elongate member 112, or they can be integrally formed with the elongate member 112. The actuators 122a-122e can also be coupled to one another to form an elongate tubular member, thereby eliminating the need for the flexible member 112. A person skilled in the art will also appreciate that, while five actuators 122a-122e are shown, the pump 110 can include any number of actuators (e.g., ranging from two to more than five). For example, the number and position of the actuators can be varied to control the flow and/or pressure characteristics of the pump and the pumping action. The actuators 122a-122e can also have a variety of configurations, shapes, and sizes to alter the pumping action of the device.

As shown, the actuators 122a-122e can be coupled to the flexible elongate member 112 in a variety of orientations to achieve a desired fluid movement. In an exemplary embodiment, the orientation of the actuators 122a-122e is arranged such that the actuators 122a-122e will radially contract and axially expand upon the application of energy thereto. In particular, when energy is delivered to the actuators 122a-122e, the actuators 122a-122e can decrease in diameter, thereby decreasing an inner diameter of the elongate member 112. Such a configuration allows the actuators 122a-122e to be sequentially activated to pump fluid through the elongate member 112, as will be discussed in more detail below. A person skilled in the art will appreciate that various techniques can be used to deliver energy to the actuators 122a-122e. For example, each actuator 122a-122e can be coupled to a return electrode and a delivery electrode that is adapted to communicate energy from a power source to the actuator. The electrodes can extend through the inner lumen 18 of the elongate member 112, be embedded in the sidewalls of the elongate member 112, or they can extend along an external surface of the elongate member 112. The electrodes can couple to a battery or other energy source. Where the pump 110 is adapted to be implanted within the patient, the electrodes can be coupled to a transformer that is adapted to be subcutaneously implanted and that is adapted to store energy and/or receive energy from an external source located outside of the patient's body. An exemplary configuration is shown in FIG. 5, in which the transformer or power source is contained in the sensor housing 60 and sensor/power leads 101 deliver energy to the pump 110.

Alternatively, energy can be supplied by an external device (e.g., the reading device 70 shown in FIG. 1A) that can transcutaneously deliver energy to the sensor housing 60 (FIG. 5), e.g., when the external device is moved in proximity of the sensor housing 60. The external device can be mobile (e.g., a wand or hand-held unit that can be waved or otherwise placed in proximity of the sensor housing 60) or stationary (e.g., a bedside, desk-mounted, or car-mounted box that the patient can move near).

FIGS. 6B-6G illustrate one exemplary method for sequentially activating the actuators 122a-122e to create a peristaltic-type pumping action. In this exemplary embodiment the pump moves fluid in a distal direction toward coil FIG. 1B), which would be located distally of the pump 110. The sequence can begin by delivering energy to a first actuator 122a such that the actuator constricts a portion of the elongate member 112 and reduces the diameter of the inner lumen 118. While maintaining energy delivery to the first actuator 122a, energy is next delivered to a second actuator 122b adjacent to the first actuator 122a. The second actuator 122b radially contracts, i.e., decreases in diameter, to further compress the elongate member 112, as illustrated in FIG. 6C. As a result, fluid within the inner lumen 118 adjacent to actuators 122a and 122b will be forced in the distal direction toward the distal end 116 of the elongate member 112. As shown in FIG. 6D, while maintaining energy delivery to the second actuator 122b, energy delivery to the first actuator 122a can be terminated, thereby causing the first actuator 122a to radially expand and return to an original, deactivated configuration. Energy can then be delivered to a third actuator 122c adjacent to the second actuator 122b to cause the third actuator 122c to radially contract, as shown in FIG. 6E, further pushing fluid through the inner lumen 118 in a distal direction. Energy delivery to the second actuator 122b can then be terminated such that the second actuator 122b radially expands to return to its original, deactivated configuration, as shown in FIG. 6F. Energy can then be delivered to a fourth actuator 122d, as shown in FIG. 6G, to radially contract the fourth actuator 122d and further pump fluid in the distal direction. This process of sequentially activating and de-activating adjacent actuators is continued resulting in a “pulse” which travels from the proximal end 114 of the pump 110 to the distal end 116 of the pump 110. The process illustrated in FIGS. 6B-6G can be repeated, as necessary, to continue the pumping action. For example, energy can be again delivered to actuators 122a-122e to create a second pulse. One skilled in the art will appreciate that the second pulse can follow directly behind the first pulse by activating the first actuator 122a at the same time as the last actuator 122d, or alternatively the second pulse can follow the first pulse some time later. One skilled in the art will further appreciate that the sequence described above can be reversed to effect flow in a proximal direction, i.e., away from the coil.

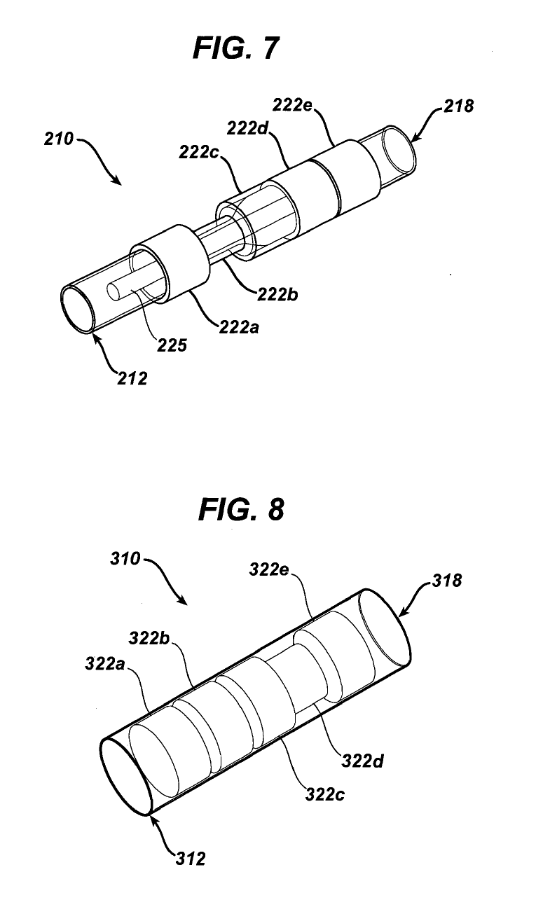

The pump 110 can also include one or more actuators configured to form a valve. By forming a valve using one or more of the actuators, the pressure and volume of fluid in the coil 20 (FIG. 1B) can be maintained without the need for additional components. In one embodiment, not shown, at least one of the actuators 122a-122e is adapted to form a valve by constricting a portion of the elongate member 112 to an extent sufficient to prevent fluid flow through the inner lumen 118. In this embodiment the elongate member 112 is constricted by one or more of the actuators 122a-122e until the inner walls of the elongate member 112 are in contact (or close proximity) with each other, preventing fluid flow through the compressed segment and thus serving as a valve. FIGS. 7-9 show various other embodiments in which the actuators can form a valve.

In the embodiment shown in FIG. 7, the pump 210 includes a plurality of actuators 222a-222e, which are formed around elongate member 212, and a reaction surface 225, which can be formed from a non-compressible material, disposed within the elongate member 212. In a contracted state, actuators 222a-222e compress the elongate member 212 against the reaction surface 225, thereby sealing the inner lumen and forming a valve. Similar to the actuators discussed above with respect to FIGS. 6B-6G, the actuators 222a-222e are in a non-compressed state in their relaxed or natural configuration, thus allowing a portion of the lumen within the elongate member 212 to remain open. The actuators 222a-222e contract when energy is delivered thereto. Using the same method described above, the actuators can be sequentially activated to create a peristaltic-type pumping action. When energy is delivered to one or more of the actuators, e.g., actuator 222b as shown in FIG. 7, the actuator will compress the elongate member 212 against the reaction surface 225 thereby preventing fluid flow through the compressed segment and thus serving as a valve. One skilled in the art will appreciate that the actuators can alternatively be configured such that in their relaxed state the actuators are closed and compress the elongate member 212 against the reaction surface 225. Upon the delivery of energy to such actuators, the actuators will expand and allow fluid to pass through the affected segments of the lumen 218. One skilled in the art will further appreciate that the sequence of activating the actuators can be controlled to effect flow in a proximal direction, i.e., away from the coil, or in a distal direction, i.e., towards the coil.

In another embodiment, shown in FIG. 8, the pump 310 includes actuators 322a-322e that are disposed entirely within elongate member 312. In this embodiment, the actuators 322a-322e can be in the form of solid members that can be of a variety of shapes, including a disk-like shape as shown. Further, the actuators are configured to radially expand or contract when energy is delivered thereto. For example, as shown in FIG. 8, the actuators can be in an expanded form in their relaxed state. In such a relaxed state the actuators occlude the lumen 318 within the elongate member 312 and prevent passage of fluid through the affected segment of the lumen. However, when energy is applied, such as to actuator 322d in FIG. 8, the actuator compresses to allow fluid to flow between an outer surface of actuator 322d and an adjacent inner surface of elongate member 312. Thus, fluid can flow past the actuators 322a-322e in their contracted state, but the actuators 322a-322e form a valve that prevents fluid flow when they are in their expanded state. A peristaltic-type pumping action can also be created by sequentially activating and deactivating the actuators 322a-322e in the manner discussed above. One skilled in the art will also appreciate that the pump of FIG. 8 can be alternatively configured such that the actuators are in a compressed configuration when in their natural state, thus allowing fluid flow, and in an expanded configuration when energy is applied thereto. One skilled in the art will further appreciate that the sequence of activating the actuators can be controlled to effect flow in a proximal direction, i.e., away from the coil, or in a distal direction, i.e., towards the coil.

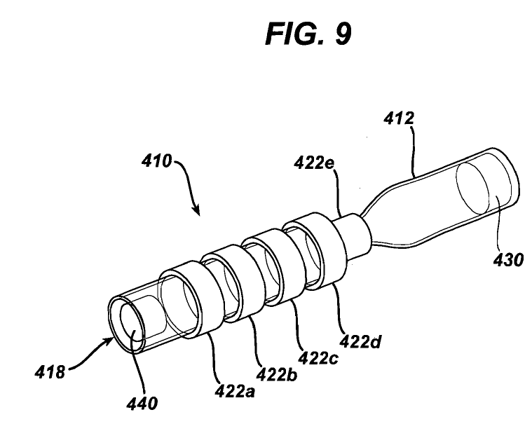

In yet another embodiment, shown in FIG. 9, the pump 410 can include both internal and external actuators. As shown, the pump 410 includes substantially solid actuators 430, 440 contained entirely inside the elongate member 412, and annular actuators 422a-422e formed on the outer surface of the elongate member 412. Any number of internal and external actuators 422a-422e, 430, 440 may be provided, and the actuators can be formed in any configuration. For example, the external actuators 422a-422e can be located between a pair of internal actuators 430, 440, which may form terminal ends of the pump. In this configuration, the internal actuators 430, 440 can operate as valves as described in detail above. For example, one of the internal actuators, e.g., actuator 430 as shown in FIG. 9, can be in an expanded form in its relaxed state to seal the inner lumen of the elongate member 412 at one terminal end of the pump. Using methods similar to those described above, the actuators can be sequentially activated to create a peristaltic-type pumping action. For example, in the configuration shown in FIG. 9, the external actuators 422a-422e can be sequentially activated and de-activated, as described above, resulting in a fluid “pulse” that travels through the inner lumen 418 of the elongate member 412. The sequential activation of the external actuators can be repeated, as necessary, to continue the pumping action. One skilled in the art will appreciate that other arrangements and configurations of internal and external actuators are possible. For example, an internal actuator 430, 440 may be located between each pair of external actuators 422a-422e. In addition, one skilled in the art will appreciate that the sequence of activating the actuators can be controlled to effect flow in a proximal direction, i.e., away from the coil, or in a distal direction, i.e., towards the coil.

One skilled in the art will appreciate that while the actuators are discussed in terms of an ability to contract and expand radially, they can alternatively be configured to contract and expand axially.

As discussed above, a fluid reservoir need not be present in the system. Instead, the elongate member 112, 212, 312, 412 shown in FIGS. 6A-9 could also serve as means for holding an additional supply of fluid. For example, the upstream (proximal) side of the elongate member 112, 212, 312, 412 can be oversized and filled with an additional volume of fluid to meet the operational needs of the system. Activation of one or more of the actuators, as discussed above, can cause fluid to move either toward or away from a coil, which would be disposed distally of the pump. One skilled in the art will appreciate that, in the embodiment discussed above in which the expansion and contraction of actuators is used to increase or decrease internal pressure within the coil without actually moving fluid, an additional volume of fluid is not needed to effect a pressure change in the system 10.

Additional information on EAP pump technology is also disclosed in commonly-owned U.S. Patent Application Publication No. 2007/0025868 A1, entitled “Electroactive Polymer-Based Pump,” filed on Jul. 28, 2005, which is hereby incorporated by reference in its entirety.

The present invention also provides a method of adjusting pressure in an implantable distension device system 10. In one embodiment, the method can include sensing a clinically relevant parameter and adjusting a pressure within the distension device in response to the sensed clinically relevant parameter by activating a pump in fluid communication with the distension device 20. The EAP-based pump can be the type described with respect to FIGS. 6A-9. That is, the pump can be formed of a plurality of actuators configured to change shape upon the application of energy thereto such that sequential activation of the plurality of actuators is effective to create pumping action to move fluid through the pump. The clinically relevant parameter can be sensed using an implantable sensor.

In one embodiment, the sensed clinically relevant parameter is a pressure, although it is understood that it can include any one of the other parameters identified above, as well as other clinically relevant parameters. In this embodiment, the pressure can be sensed using an implantable pressure sensor 62, as discussed above. The method can include sensing a pressure in an implanted distension device 10a, comparing the sensed pressure to a desired pressure (including a desired pressure range), and adjusting the pressure within the distension device 10a to be approximately equal to the desired pressure (or desired pressure range) if the sensed pressure is not equal to the desired pressure (or desired pressure range) by activating a pump in fluid communication with the distension device 20 to achieve a desired pressure (or desired pressure range) in the distension device.

In one embodiment, activation of the pump 110 could automatically occur if the sensed clinically relevant parameter (e.g., pressure, etc.) in the coil 20 were higher than a desired value or range, in which case fluid could be pumped out of the coil 20 to reduce the pressure. Conversely, if the sensed parameter in the coil 20 were lower than a desired value or range, the fluid could be pumped into the coil (e.g., from a reservoir or from an implanted catheter) until a desired target for the parameter is achieved. It is understood that depending on what is being sensed, and where it is being sensed, the decision to pump fluid into or out of the coil bladder in response to a give level of the clinically relevant parameter may be reversed. In yet another configuration, if a sensed clinically relevant parameter (e.g., absolute pressure at a given duration, pressure gradient, etc.) in the coil 20 which correlates with undesirable eating habits was measured, the fluid could be pumped into the coil (e.g., from a reservoir or from an implanted catheter) until a sufficient distension was created. This distension would provide feedback to the patient (which can be immediate or delayed) to stop eating by inducing a physiologic response (e.g., vomiting, etc.). The distension would be sustained in place until a triggering event (e.g., elapsed time) occurred to return the system to a normal operating state. For safety purposes, an override which can be activated by the patient or other caregiver may be provided. This override may be activated through a function in the external portion 10b of the stomach distension system 10. Other techniques for automatic actuation can be used such as timer control, or the system can be programmed to activate the pump in response to certain sensed parameters or events, or according to a programmed schedule. For example, the implantable pump can effect a pressure increase within the distension device (i.e., move fluid towards the distension device) when a patient is determined to be eating, or when the patient is awake (or during selected hours of a day) and effect a pressure decrease within the distension device (i.e., move fluid away from the distension device) when the patient is asleep (or during other selected hours of a day). Those skilled in the art will appreciate that the programmed schedule can be based on a multitude of factors including type of day (e.g., holidays, weekday, weekend), anticipated patient activities, and the like. Those skilled in the art will appreciate that the pressure in the coil 20 can be controlled using closed-loop methods such as PID (proportional-integral-derivative) control schemes or other appropriate methods including digital control schemes.

One skilled in the art will appreciate that certain safety features may be built into the pump design to provide contingencies in the event of a malfunction or a loss of power. By way of example, if a power outage (or malfunction) is detected, or if the remaining power falls below a predetermined threshold, the system can be configured to default to a relaxed state in which the distension is relaxed and/or opened until the power level is restored or the malfunction corrected.

An alternative pump may be similar to implantable insulin pumps that are commonly used. Displacement of the piston draws the fluid from a reservoir into a piston chamber; when the piston returns to its original position, it forces the fluid through a free floating catheter, which is inserted into the coils bladder. The fluid pump uses freon gas to produce positive pressure, which pushes the fluid from a reservoir into a valve-type accumulator and into the catheter. The reservoirs in would be refillable. A hypodermic needle, inserted directly through the patient's skin into the pump's reservoir, removes any unused fluid and replaces it with a fresh supply. The Hypodermic needle may also access the pumps reservoir in a Trans oral if the reservoir is inside the stomach.

There is an opportunity to increase the output from the pump by using a hydraulic amplifier or intensifier. This is defined as A fluid device which enables one or more inputs to control a source of fluid power and thus is capable of delivering at its output an enlarged reproduction of the essential characteristics of the input. Hydraulic amplifiers may utilize sliding spools, nozzle-flappers, jet pipes, etc.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized by any known and suitable technique, including ethylene oxide sterilization. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that device is sterilized. This can be done by any number of ways known to those skilled in the art including beta or gamma radiation, ethylene oxide, steam.

Any patent, publication, application or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device, including an implant for placement within a hollow body organ, said device comprising:
a. a distension device having an undeployed shape for delivery within a hollow body and one or more deployed shapes for implantation therein;
b. said distension device having sufficient rigidity in its deployed shape to exert an outward force against an interior of the hollow body so as to bring together two substantially opposing surfaces of said hollow body; and
c. an implantable pump in fluid communication with the distension device and having a plurality of actuators configured to change the shape of said distension device upon the application of energy thereto such that sequential activation of the plurality of actuators is effective to create pumping action to move fluid through the pump.

2. The device of claim 1, further comprising an implantable port configured to receive fluid from a fluid source external to the patient wherein the implantable port is in fluid communication with the implantable distension device and the pump.

3. The device of claim 1, further comprising an implantable sensor in communication with the distension device.

4. The device of claim 3, wherein the implantable sensor is configured to measure at least a pressure within the distension device.

5. The device of claim 1, wherein the pump further comprises a first member having a passageway formed therethrough and being in communication with the plurality of actuators.

6. The device of claim 5, wherein the actuators are disposed within the first member.

7. The device of claim 5, wherein the actuators are disposed outside the first member.

8. The device of claim 1, wherein at least one actuator is configured to expand upon the application of energy thereto.

9. The device of claim 1, wherein at least one actuator is configured to contract upon the application of energy thereto.

10. The device of claim 1, wherein the actuators are configured to move sequentially.

11. The device of claim 1, wherein each actuator comprises an electroactive polymer.

12. The device of claim 1, wherein fluid moves in a direction from the pump to the distension device.

13. The device of claim 1, wherein fluid moves in a direction from the distension device to the pump.

14. The device of claim 1, wherein the implantable pump effects a pressure change within the distension device in accordance with at least one of a detected event and a programmed schedule.

15. The device of claim 1, further comprising a fluid reservoir in fluid communication with the pump.

16. The device of claim 15, wherein the fluid reservoir is configured to hold in the range of approximately 0.1 to 20 ml of fluid.

17. The device of claim 1, wherein at least one of the actuators serves as a valve that is able to selectively control the passage of fluid by permitting, preventing, or limiting the passage of fluid.

18. A method of adjusting pressure in an implantable distension device, comprising:
a. providing a distension device having an undeployed shape for delivery within a hollow body and one or more deployed shapes for implantation therein, said distension device having sufficient rigidity in its deployed shape to exert an outward force against an interior of the hollow body so as to bring together two substantially opposing surfaces of said hollow body, an implantable pump in fluid communication with the distension device and having a plurality of actuators configured to change the shape of said distension device upon the application of energy thereto such that sequential activation of the plurality of actuators is effective to create pumping action to move fluid through the pump sensing a clinically relevant parameter;
b. sensing a clinically relevant parameter within a body; and
c. adjusting a pressure within the distension device in response to a by activating a pump in fluid communication with the distension device.

19. The method of claim 18, wherein the step of sensing a clinically relevant parameter within a body comprises measuring pressure in the implantable distension device.

20. The method of claim 18, wherein said step of adjusting a pressure within the distension device is automatically activated.

21. The method of claim 18, further including the step of providing feedback to a patient.

22. The method of claim 19, further comprising the steps of comparing the sensed pressure to a desired pressure range and adjusting the pressure within the distension device to be within the desired pressure range.

23. A pumping device, comprising:
a. a fluid conduit member having a passageway formed therethrough; and
b. a plurality of orientation-changing actuators disposed within the fluid conduit member, each actuator being independently configurable between a normal, relaxed state in which the actuator occludes a portion of the passageway of the fluid conduit member and an energized configuration in which fluid flow is permitted between an outer surface of the actuator and an inner surface of the fluid conduit member, the actuators being configured to change orientation upon the application of energy thereto such that sequential activation of the plurality of actuators is effective to create pumping action to move fluid through the first member.

24. The device of claim 23, wherein each actuator comprises an electroactive polymer.

25. The device of claim of claim 23, wherein each actuator comprises a least one electroactive polymer composite having at least one flexible conductive layer, an electroactive polymer layer, and an ionic gel layer.

26. The device of claim of claim 23, wherein each actuator includes a return electrode and a delivery electrode coupled thereto, the delivery electrode being adapted to deliver energy from an energy source.

27. The device of claim of claim 23, wherein the actuators are configured to move independently.
